(19) United States
(12) Patent Application Publication
(10) Pub. No.: US 2021/0177014 A1
(43) Pub. Date: Jun. 17, 2021

(54) METHOD FOR THE THERMAL TREATMENT OF BULK MATERIALS IN A ROTARY TUBE WITH AT LEAST ONE INFRARED LIGHT UNIT

(71) Applicant: Kreyenborg GmbH & Co. KG, Senden (DE)

(72) Inventors: Siegfried KRAUSS, Meiningen (DE); Michael FUHRMANN, Rippershausen (DE)

(73) Assignee: Kreyenborg GmbH & Co. KG, Senden (DE)

(21) Appl. No.: 17/191,265

(22) Filed: Mar. 3, 2021

Related U.S. Application Data
(63) Continuation of application No. PCT/DE2019/100791, filed on Sep. 3, 2019.

(30) Foreign Application Priority Data
Sep. 3, 2018 (DE) 10 2018 121 453.7

(51) Int. Cl. A23L 3/005; F26B 3/30; F26B 11/02; A23L 3/16; A23L 3/54; A23L 3/00; B29C 71/02

(52) U.S. Cl. CPC A23L 3/0055; F26B 3/30; F26B 11/02; F26B 11/026; A23B 7/01; A23L 3/54; A23L 3/001; B29C 71/02; A23V 2002/00; A23L 3/165

(57) ABSTRACT

A method for the thermal treatment of bulk materials in a rotary tube with at least one infrared light unit. Bulk material is introduced into the rotary tube, which is provided on its inner wall with at least one mixing element and in the interior space of which the pressure of the ambient atmosphere prevails. A heat treatment of the bulk material is performed by at least one electrical infrared light unit, which is arranged at the center of the rotary tube and the light cone of which is directed onto the bed of bulk material that lies on the inner wall of the rotary tube. The bulk material is discharged from the rotary tube. Water vapor is directed onto the surface of the bulk material. The vapor is introduced into the interior space of the rotary tube through a nozzle tube.

This nonprovisional application is a continuation of International Application No. PCT/DE2019/100791, which was filed on Sep. 3, 2019 and which claims priority to German Patent Application No. 10 2018 121 453.7, which was filed in Germany on Sep. 3, 2018 and which are both herein incorporated by reference.

### BACKGROUND OF THE INVENTION

### Field of the Invention

The present invention relates to a method for the thermal treatment of bulk materials in a rotary tube with at least one infrared light unit.

### Description of the Background Art

Such a method is described in DE 10 2013 223 929 A1 (which corresponds to US2016/0255871, which is incorporated herein by reference) and WO 2015/067255 A1. Accordingly, an infrared rotary dryer is very well suited for reducing the germ count in dried raw foodstuffs, such as for example seeds, vegetables, herbs, spices, mushrooms, tea, nuts and dried feed. The use of the rotary tube allows both batch operation and run-through operation. This effect is enhanced in particular by the periodic spraying in of water. The product, which is permanently kept moving in the rotary tube by the rotation and additionally by the mixing elements reaching into the bed of bulk material, is very quickly brought to a defined temperature by the heat of the infrared light and kept at this temperature for a specific time. At the same time, a fine water mist or saturated steam may be sprayed in at this temperature level. This method is very effective for sterilizing many foods, since the adding of water has the effect that a longer treatment time is possible and the sterilizing and pasteurizing effect is promoted to a greater extent than would be possible just by heating with infrared light. This is so because, in the latter case, surface temperatures of 140° to 170° C. can occur, which may cause instances of burning on the surface of the product. By adding water, the thermal energy introduced is not only brought to the surface of the food particles that are exposed to the infrared light but also into layers lying thereunder. The feeding in of water has the effect of limiting the temperature at the product to approximately 135° C., whereby overheating is avoided. This temperature lies in the range of the ultrahigh heating that is used for example for pasteurizing milk. However, it has been found that the temperature mentioned is not always sufficient to kill spore-forming bacteria, in particular on coarsely porous substrates such as black pepper.

A temperature increase that is possible in principle by means of a significant increase in pressure in an autoclave, as used in steam sterilizers and the like in the medical sector, does not however come into consideration for large-scale industrial use in the drying and heat treatment of large quantities of bulk materials for economic reasons.

### SUMMARY OF THE INVENTION

It is therefore an object of the present invention to achieve effective decontamination of bulk materials with regard to spore-forming bacteria by using the known partly open rotary tube, and consequently without a significant difference in pressure in relation to the ambient atmosphere.

This object is achieved according to an exemplary embodiment of the present invention by a method that introduces steam, specifically in a specific zone. According to the invention, the steam is not fed directly into the bulk material, but just above it. Furthermore, the outlet nozzles are placed such that the steam flows out in the cone of light of the infrared light units. The choice of steam instead of water and the placement of the steam nozzles are the essential features of the invention.

In the interior space, substantially the normal ambient air pressure prevails. It is possible for local increases in pressure to occur during the feeding in of the steam. However, because of the non-closed type of construction, pressure in the interior space always equalizes again to the ambient conditions.

Spraying in steam instead of water eliminates the heat losses occurring in the conventional art as a result of the enthalpy of evaporation of the water, which is a cause of the aforementioned temperature limitation of the conventional process.

The positioning of the steam nozzles in the infrared light achieves the effect that the outflowing steam in the infrared light is brought to a higher temperature level, specifically already while it is flowing through the steam line, since the steam line is also in the cone of light, so that the steam line is already heated up and in this way the steam carried in it is heated up. The further heating up takes place all the more once the steam flows out of the steam nozzles in the direction of the bulk material. Therefore, an “overheating” takes place in the sense that, by the method according to the invention, the steam is hotter than it would be theoretically after leaving a steam line that is not additionally heated, and after the accompanying expansion and drop in pressure. In order however to create a distinction from the technical term of superheated steam, in the following description of the method according to the invention reference is made to after-heating.

The introduction of after-heated steam has the effect that the decontaminating effect is significantly enhanced, and in particular the killing of spore-forming germs is achieved when treating foods in bulk form, such as herbs, spices, fruits, nuts, seeds, tea, mushrooms and/or roots. In particular in the case of spices known to be highly contaminated with germs such as black pepper and vanilla pods, effective sterilization is achieved with relatively short run-through times.

The after-heated steam heats up abruptly to high temperatures when it meets the surface of the bulk material particles. At the same time, the specific heat capacity of the quantity of steam that comes into contact with the surface of the bed of bulk material is relatively small in comparison with the specific heat capacity of the treated solid particles, so that a cooling down of the steam, and possibly also to a slight extent a condensation, takes place when the steam enters the bed of bulk material. The after-heated steam therefore only has a superficial effect, but does not however bring about a deeper disadvantageous overheating of the product.

A further effect of the steam is the displacement of oxygen, so that as it were an inert atmosphere is created in the region of the introduction of the steam. Oxygen promotes burning of the product. The displacement of the oxygen by the steam can have the effect of achieving greater decontamination temperatures, since the oxygen concentration in the product bed is very small and instances of burning of the product are prevented.

Since superheated steam is free from air, and consequently from oxygen, it can also be used for oxygen-sensitive products that are otherwise treated for example in a nitrogen atmosphere.

The use of the infrared rotary tube with the internal mixing elements has the effect that changing layers of the bulk material are always exposed to the influence of the hot steam in addition to the infrared light. This therefore repeatedly achieves instances of local heating, with high temperatures of up to 190° C., in the layers of the bulk material particles near the surface in which the bacteria reside, while subsequently however—if after the recirculation the particles that were previously under the influence of the steam lie in lower layers of the bed of bulk material again—cooling down to a level at which no damaging effects to the product occur is achieved very quickly.

The steam fed in and the infrared light units provide two independent heat sources which in run-through operation can be used in different ways axially one after the other and/or at times one after the other or at the same time, and in batch operation can be used at the same time or at times one after the other.

In each case, the bulk material may first be brought to a temperature level beyond the boiling point of water, or at least very close to it, exclusively by the infrared light units. This avoids excessive condensation of steam on contact with the bulk material, since intensive humidification would lead again to great heat losses in the subsequent further course of the heat treatment because of the enthalpy of evaporation of the water, which would lead to the already described limitation to an insufficient level of the process temperature that is effective on the bulk material. The preheating of the bulk material with infrared light before the beginning of the feeding in of the steam avoids this.

In order to combat very stubborn germs, it may be advantageous to feed in saturated or even superheated steam in the known technical sense, water therefore only being in the gaseous state of aggregation and free from water droplets. The steam flows out of the steam nozzles onto the bed of bulk material and rises up from there in the rotary tube, where it is possibly extracted in order to avoid condensation in the interior space of the rotary tube. The flow path of the steam lies completely, or at least mostly, in the cone of light of the infrared light units. On account of the overheating of the steam that already occurs from the outset, and a continuing supply of heat after it leaves the nozzles, the superheated steam remains free from water mist, and intensive condensation on the bulk material can be avoided.

A further effect of the treatment of products with infrared light and after-heated steam is that extraneous odor is drastically reduced or their own characteristic, unpleasant odor is removed entirely.

It has been found that the method according to the invention, which was originally designed for the decontamination of foods, is also very effective for the treatment of bulk material particles of plastic in the recycling process, in particular for the elimination of extraneous odor and their own odor that originates from attached organic-aromatic substances, contains volatile contaminants due to migrated substances. Treated as bulk material may be in particular particles of thermoplastics, thermoplastic elastomers and vulcanizates (TPE/TPU/TPE-V) that contain residues of monomers and oligomers or other volatile contaminants due to migrated substances.

An example of this is the typical smell of gasoline or diesel in the case of fuel canisters or tanks made of plastic. The method according to the present invention is therefore also suitable for eliminating attached matter in the recycling of such plastic containers without melting the plastic. During the processing of particles of plastic, more intensive condensation may be deliberately made possible in the process than in the case of food treatment, so that the detached substances can be precipitated in aqueous solution.

A device for carrying out the method provides, in addition to the rotary tube with at least one infrared light unit, which is installed in the clear cross section in the interior space of the rotary tube, that is to say well away from the wall, the following:

A steam-inflow device with multiple steam nozzles is positioned in the cone of infrared light in a fixed, pivotable, slidable or foldable manner, so that the steam flowing through the cone of light is after-heated.

A steam-inflow device in the form of a steam lance can be adjusted to different lengths in order to allow different inflow times in the continuous rotary tube.

Segments can be shut off, in order to define different phases of the steam treatment and to realize different exposure times to the steam in a continuous rotary tube.

The so-called steam lance, which bears the steam nozzles, can be arranged in a lower region of the rotary tube. On account of the rotation and the friction with the inner wall, the bed of bulk material in the rotary tube rises up in a sloping position, that is to say that, during the rotation, the middle of the bed of bulk material is not in a 6 o'clock position but rather at 6 to 8 o'clock or 4 to 6 o'clock, depending on the direction of rotation and viewing direction. The steam lance is then preferably mounted at the edge of the bed of bulk material in the lower 6 o'clock position. The steam rises up from there.

It is very advantageous in the method according to the invention to use a so-called air shield and to direct its air flow onto the bed of bulk material. This is a powerful blower, which usually protects the infrared light units from local overheating and soiling with dirt particles from the treated bulk material. Directing the air flow of the air shield onto the surface of the bed of bulk material causes the steam that leaves the steam lance to be forced onto the sloping surface of the bed of bulk material, instead of rising up vertically. Consequently, the effectiveness of the steam for the treatment of the bulk material is increased significantly.

With regard to how the method is conducted, an adaptation to the product that is respectively being treated can be performed in particular by the following parameters or measures discussed below.

In continuous run-through operation, first a heating up by infrared light alone takes place at the entry of the rotary tube, before a steam treatment takes place over a longer axial distance.

With simultaneous exposure to infrared light and steam, the product is kept at a temperature between 90° C. and 220° C. for a time between 1 min and 25 min, in particular at 150° C. to 200° C. for 10 min to 20 min.

Phases of the combined treatment with infrared and steam and phases of just infrared light treatment may alternate over the axial extent in the rotary tube; the two heat sources are to be operated independently of one another, at least in some sections.

Before and/or after the inflow of steam, the product may be subjected to a water mist. This requires a careful appraisal of the nature of the product being treated. In the case of plastics, for instance, a water mist is advantageous because high temperatures in the process are not as important as complete separation of attached matter and because the unwanted substances can be advantageously discharged in aqueous solution. In the case of foods, on the other hand, applied water can evaporate in the infrared light and lead to a steam shock effect. If water is applied at the end of the treatment, it brings about a cooling down and re-humidification, in order to restore the original water content.

A process sequence, given by way of example, for killing spores in vanilla or pepper envisages that the product is first heated to temperatures between 50° C. and 150° C. in the infrared rotary tube, while at the same time spraying in water for 3 min to 25 min, typically approximately 12 min. Subsequently, while steam simultaneously flows into the product bed, the product is heated with infrared light at temperatures between 100° C. and 220° C. and is kept at this temperature level under infrared light and inflow of steam for 1 min to 20 min. Subsequently, with reduced infrared light power, the product can be sprayed with a water mist and cooled down.

A process sequence, given by way of example, for drastically reducing the characteristic own odor in the plastics sector comprises heating up bulk plastics material, such as for example a thermoplastic elastomer/vulcanized (TPE-V), by means of infrared light units in the continuously charged and rotating rotary tube to a temperature between 70° C. and 150° C. Subsequently, the moved bulk material is treated in this temperature range with after-treated steam for 8 min to 25 min and subsequently cooled down.

Further scope of applicability of the present invention will become apparent from the detailed description given hereinafter. However, it should be understood that the detailed description and specific examples, while indicating preferred embodiments of the invention, are given by way of illustration only, since various changes, combinations, and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 shows an infrared rotary tube unit in a schematic sectional view;

FIG. 2 shows a diagram with a temperature profile over time according to an exemplary method, given by way of example, and

FIG. 3 shows a diagram with a temperature profile over time according to an exemplary method, given by way of example.

### DETAILED DESCRIPTION

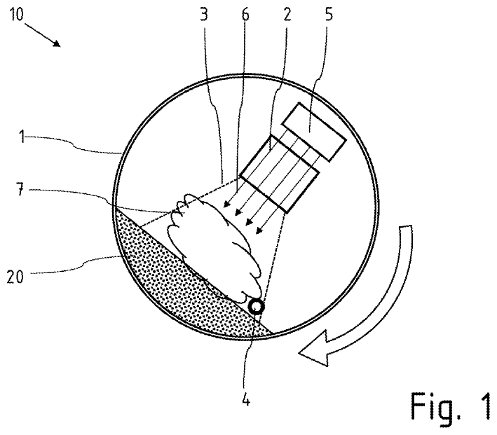

FIG. 1 shows an infrared rotary tube unit 10 in a schematic sectional view. It substantially comprises a rotatable rotary tube 1, which has a closed casing at its circumference, and, arranged in the clear cross section therein, an infrared light unit 2, which radiates infrared light in a cone of light 3. The cone of light 3 is directed onto a bed of bulk material 20, which rests on the inner wall in the lower region of the rotary tube 1. Mixing elements and conveying elements, such as a screw flight, provide constant recirculation and conveyance, but are not shown here. The direction of rotation is indicated by the block arrow. The rotation has the effect that the bed of bulk material 20 assumes a sloping alignment due to friction with the wall. Both the cone of light 3 and an air flow of an air shield 5 indicated by the arrows 6 are directed perpendicularly onto the surface of the bulk material. Steam 7 leaves from a steam lance 4, which lies in the cone of light and has multiple steam nozzles over its length. The arrangement of the steam lance 4 with respect to the direction of rotation is important because the steam lance 4 should be arranged such that it is positioned at the lower edge of the bed of bulk material 20 resting on the inner wall of the rotary tube 1. Consequently, after leaving the steam lance 4, the steam automatically passes over the bed of bulk material 20. Preferably, the air flow 6 of the air shield 5 additionally forces the emerging steam 7 onto the surface of the bed of bulk material 20 and prevents the steam 7 from rising vertically upward on account of its significantly higher temperature, and consequently lower density, in comparison with the air temperature inside the rotary tube 1.

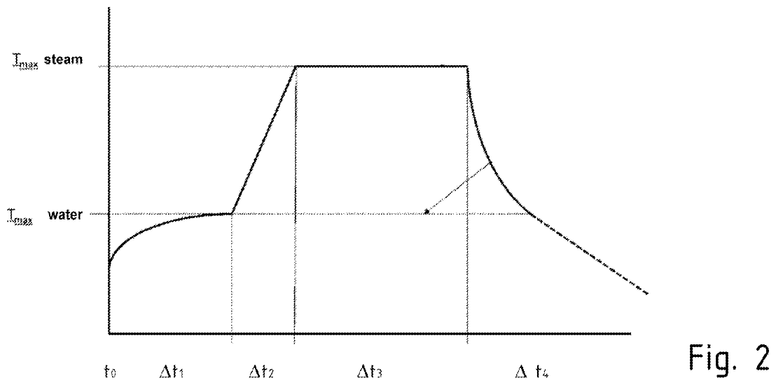

In the diagram that is shown in FIG. 2, the temperature is plotted over time according to a first way of conducting the method, given by way of example.

Beginning at the time to, the product is heated in a time phase Δt₁ up to a base temperature. In a subsequent time phase Δt₂, a further rise in the temperature is achieved by the spraying in of steam. After the time phases Δt₁, Δt₂, the heating-up phase is ended and this is followed by the actual treatment phase over a time phase Δt₃, in which the high temperature is maintained. The comparison shows that the treatment temperature Tₘₐₓ (steam) achievable by spraying in steam is higher than the holding temperature achieved by the known method, which lies at the level Tₘₐₓ (water). The cooling-down time Δt₄ from the high temperature level Tₘₐₓ (steam), measured from the ending of the supply of steam and switching off of the infrared light, is not much greater in comparison with the cooling down from the lower temperature level Tₘₐₓ (water), because the process according to the invention especially has the effect that the surface is heated up much more, but the core of the product is heated up much less.

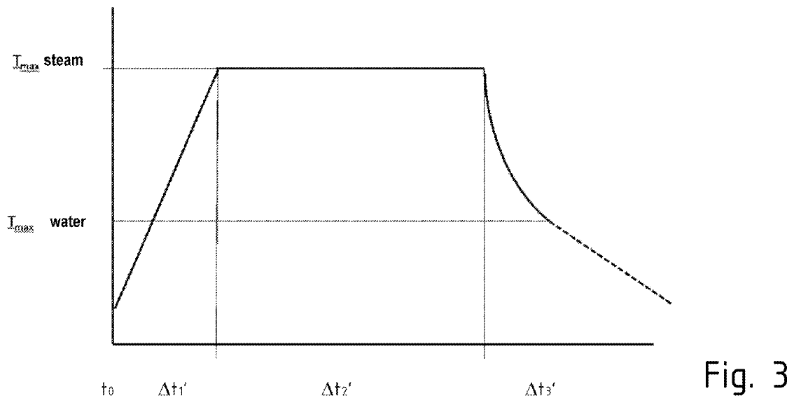

In FIG. 3, the temperature is plotted over time according to another way of conducting the method, given by way of example. Here, the product is subjected to steam from the time to, and a much higher peak temperature in comparison with the prior art is already achieved within a short time span Δt₁′.

In the case of both variants of the method of the invention, the product is not damaged in spite of the much higher final temperature in the layers near the surface as a result of the additional spraying in of steam.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

What is claimed is:

1. A method for thermal treatment of bulk materials in a rotary tube with at least one infrared light unit, the method comprising:
introducing bulk material into the rotary tube, which is provided on its inner wall with at least one mixing element and in an interior space of which a pressure of the ambient atmosphere prevails;
carrying out a heat treatment of the bulk material by at least one electrical infrared light unit, which is arranged in a center of the rotary tube and the cone of light of which is directed onto the bed of bulk material, which rests on the inner wall of the rotary tube;
discharging the bulk material from the rotary tube;
directing steam for the heat treatment onto the surface of the bulk material;
introducing the steam into the interior space of the rotary tube through at least one nozzle tube provided with multiple steam nozzles;
arranging the nozzle tube with its steam nozzles in the cone of light of the infrared light unit and outside a cross section of the interior space of the rotary tube covered by the bulk material; and
after-heating the steam by the infrared light unit within the part of the flowed-through nozzle tube that is located in the cone of light beyond its exit temperature at the steam nozzles.

2. The method as claimed in claim 1, wherein the radial distance of the steam nozzles from the bulk material is 0.1 times to 2.0 times the screw flight height of a screw flight mounted on the inner wall of the rotary tube.

3. The method as claimed in claim 1, wherein the temperature of the steam at the surface of the bed of bulk material is more than 140° C.

4. The method as claimed in claim 2, wherein steam superheated by way of the steam nozzles is introduced.

5. The method as claimed in claim 1, wherein, in addition to the steam, water is directed onto the bed of bulk material, and wherein the outlet nozzles of a water line are arranged above or below the cone of light of the infrared light unit.

6. The method as claimed in claim 1, wherein an airshield, the air flow of which is directed onto the bed of bulk material is provided at the infrared light unit, and wherein the cone of light and the air flow of the airshield are directed substantially perpendicularly onto the surface of the bulk material.

7. The method as claimed in claim 1, wherein food in the form of bulk material is used as the bulk material.

8. The method as claimed in claim 1, wherein particles of plastic tainted with organic-aromatic and/or other chemical compounds are treated as the bulk material.

9. The method as claimed in claim 8, wherein particles of thermoplastic vulcanizates are used as the bulk material.
